(19) United States
(12) Patent Application Publication
(10) Pub. No.: US 2020/0009238 A1
(43) Pub. Date: Jan. 9, 2020

(54) ENGINEERED INVARIANT CHAIN MOLECULE FOR IMPROVED MHC CLASS I LOADING

(71) Applicant: UNIVERSITETET I OSLO, Oslo (NO)

(72) Inventors: Sebastien P. WÄLCHLI, Oslo (NO); Tone Fredsvik GREGERS, Oslo (NO); Else Marit Suso INDERBERG, Oslo (NO); Gustav GAUDERNACK, Sandvika (NO); Gunnar KVALHEIM, Oslo (NO); Oddmund BAKKE, Oslo (NO)

(21) Appl. No.: 16/582,209

(22) Filed: Sep. 25, 2019

Related U.S. Application Data
(63) Continuation of application No. 15/317,338, filed on Dec. 8, 2016.

(30) Foreign Application Priority Data
Jun. 10, 2014 (EP) 14171798.3

(51) Int. Cl. A61K 39/00; C07K 14/495; C07K 14/74; A61K 9/00

(52) U.S. Cl. CPC A61K 39/0011; C07K 14/495; C07K 14/70539; A61K 9/0009; A61K 2039/605; A61K 2039/58; C07K 2319/00; A61K 2039/53; A61K 2039/54; A61K 2039/57; A61K 2039/5158

(57) ABSTRACT

The present invention relates to peptides presented on the cell surface of cells in the MHC class I (MHC I) context in which the invariant chain has been engineered to favor loading of specific antigens and generate CD8⁺ T-cell activation

The present application is a continuation of U.S. patent application Ser. No. 15/317,338, filed Dec. 8, 2016, which is a 371 National Entry application of PCT/EP2015/062933, filed Jun. 10, 2015, which claims priority to EP 14171798.3 filed Jun. 10, 2014, each of which are incorporated by reference in their entireties.

### FIELD OF THE INVENTION

The present invention relates to peptides presented on the cell surface of cells in the MHC class I (MHC I) context in which the invariant chain has been engineered to favor loading of specific antigens and generate CD8⁺ T-cell activation. The present invention also relates to vaccine constructs where the CLIP region of the wild type li (liwt) has been replaced with different antigens to generate CD4⁺ and CD8⁺ responses. In on embodiment is these antigens generated from a frameshift mutation in TGFbRII.

### BACKGROUND OF THE INVENTION

CD4⁺ T cells generally recognize exogenously derived peptides presented on the cell surface of antigen presenting cells in the MHC class II (MHC II) context. The biosynthesis and transport of MHC II molecules are tightly regulated by the type II transmembrane invariant chain (Ii).

Ii harbors two leucine based sorting signals; Leu7/Ile8 and Met16/Leu17 in its cytoplasmic tail. Indeed, these signals direct the Ii-MHC II complex to the endosomal pathway mainly via the cell surface, (therefore called the indirect pathway) and through binding to the adaptor proteins AP-1 and AP-2. These APs are involved in the formation of clathrin-coated vesicles at the trans Golgi and plasma membrane respectively. In addition, they play a pivotal role in cargo selection by recognizing the appropriate sorting signals of integral membrane proteins. Either one of the two Ii leucine signals is sufficient for targeting Ii to endosomal compartments.

Within the endosomes, Ii is sequentially degraded leaving the class II-associated Ii peptide (CLIP) bound to the MHC II groove. CLIP is subsequently exchanged for antigenic peptides prior to transport to the cell surface for presentation. Several studies have shown that by genetically exchanging the CLIP region with antigenic peptides, MHC II molecules are efficiently loaded with the peptide and presented to specific CD4+ T cells.

Unlike MHC II, MHC I binds mainly endogenously derived peptides generated by the proteasome in the cytosol which are targeted to the endoplasmic reticulum (ER) via TAP transporters. After MHC I peptide loading, the complex is presented to cytotoxic CD8+ T cells.

MHC I was originally thought to be independent of Ii, however, it was recently demonstrated that Ii plays a physiological role for targeting MHC I to the endosomal pathway thereby enabling loading of viral and other peptides and in the endosomal pathway and presentation to CD8+ T cells. This is then called cross-presentation.

Additional evidence for an Ii-MHC I interaction came from van Luijn and colleagues who showed that CLIP efficiently binds to several MHC I molecules in leukemic cells.

Furthermore, the present inventors recently showed that when CLIP is exchanged with an MHC I tumor specific antigen, the peptide loads onto MHC I in a proteasome/TAP/tapasin independent manner.

This strategy was found to be as efficient as exogenous loading of synthetic peptide in vitro.

Thus, the data clearly corroborates an Ii influence on not only MHC II, but also on MHC I antigen presentation.

There is a need for improving this antigen presentation in order to generate improved CD8⁺ T-cell activation. This activation can benefit from additional activation of CD4⁺ cells.

### SUMMARY OF THE INVENTION

An object of the present invention is to engineer a molecule with integrated antigens that enables loading and presentation of the antigens on the surface of cells to generate CD8⁺ T-cell activation.

In one embodiment of the present invention is CD4⁺ T-cell activation also achieved.

An aspect of the present application relates to a nucleic acid molecule encoding a type II transmembrane invariant chain (Ii) which is modified by exchanging the class II-associated Ii peptide (CLIP) with an antigen, and wherein one or more sorting motifs is/are replaced with a AP-3 binding motif.

In another aspect of the present invention is Ii wildtype (Iiwt) which is modified by exchanging the class II-associated Ii peptide (CLIP) with an antigen.

In one embodiment of the present invention is the sorting motif a leucine or a tyrosine based sorting motif.

In a further aspect of the present invention is one or more sorting signals a cytoplasmic sorting motif selected from the group consisting of Leu₇/Ile₈ and Met₁₆/Leu₁₇.

In yet another aspect of the present invention is the sorting motif (QLP)L7I (SEQ ID NO:32) replaced with (RRP)L7I (SEQ ID NO:33) and/or (QRD)L17A (SEQ ID NO:34) is replaced with (RRP)L17A (SEQ ID NO:35).

In a further embodiment of the present invention is the antigen a tumor antigen.

In another embodiment of the present invention is the nucleic acid molecule selected from the group consisting of mRNA and DNA.

In another embodiment of the present invention is the nucleic acid molecule mRNA.

Another aspect of the present application relates to an amino acid molecule comprising the type II transmembrane invariant chain (Ii) according to the present invention.

Another aspect of the present application relates to a method of presenting a CD8⁺ T-cell activating antigen on a cell, comprising modifying the type II transmembrane invariant chain (Ii) by exchanging the class II-associated Ii peptide (CLIP) with an antigen, replacing one or more sorting motifs with a AP-3 binding motif, and introducing the Ii to a cell.

In one embodiment of the present invention, the antigen also activates a CD4⁺ T-cell.

Another aspect of the present application relates to a pharmaceutical composition comprising the nucleic acid molecule or the amino acid molecule according to the present invention, and a pharmaceutically acceptable carrier, excipient and/or diluent.

In one embodiment of the present invention is the pharmaceutical composition a vaccine.

Another aspect of the present application relates to a method for inducing a CD8⁺ and/or a CD4⁺ response, comprising administering the nucleic acid molecule or the amino acid molecule according to the present invention to an individual.

In one embodiment of the present invention is the nucleic acid molecule or amino acid molecule administered using electroporation.

Another aspect of the present application relates to the nucleic acid molecule or the amino acid molecule according to the present invention for use as a medicament.

A further aspect of the present application relates to a nucleic acid molecule or the amino acid molecule of the present invention for use in the treatment of a disease that is associated with the antigen.

### BRIEF DESCRIPTION OF THE FIGURES

FIG. 1A

Ii constructs used. Here showing the amino acid sequences of the Ii cytoplasmic tails. The Ii wt, sorting signal (QRD)L7I was replaced by an (RRP) L7I motif resulting in the trafficking mutant Ii RRP/L17A. For Ii L7A/L17A, both L signals were removed. Indicated are also; the trans membrane (TM) region, CLIP, the two N-glycosylation sites (113 and 119) and the trimerization domain (TRI).

FIG. 1B

Hek cells were transfected as indicated. After 24 hours, whole cell lysates (WCL) were subjected to 4-20% SDS-PAGE Tris-HEPES-SDS gels, transferred to PVDF membranes and probed with anti Ii antibody, M-B741. The samples were either boiled or non-boiled before gel loading. Ii trimers and monomers are shown. Detection of actin in the WCL was used as loading control using anti-actin antibodies.

FIG. 1C

Endo H test. Transfected Hek cells were pulsed with 35SMethionine/Cystein containing media for 30 min, washed and thereafter lysed. Ii protein was immunoprecipitated, the precipitates were split in two where one part was treated with Endo H, the other not. Three Ii fractions were detected for the treated samples representing; the light/Endo H sensitive ER fraction, the premature Endo H resistant fraction with only one glycan and the fully mature double N-linked glycosylated mature fraction of Ii. Thus, all contructs gained Endo H resistance indicating that despite the presence of the RRP amino acid sequence, regular transport from ER through the Golgi was retained.

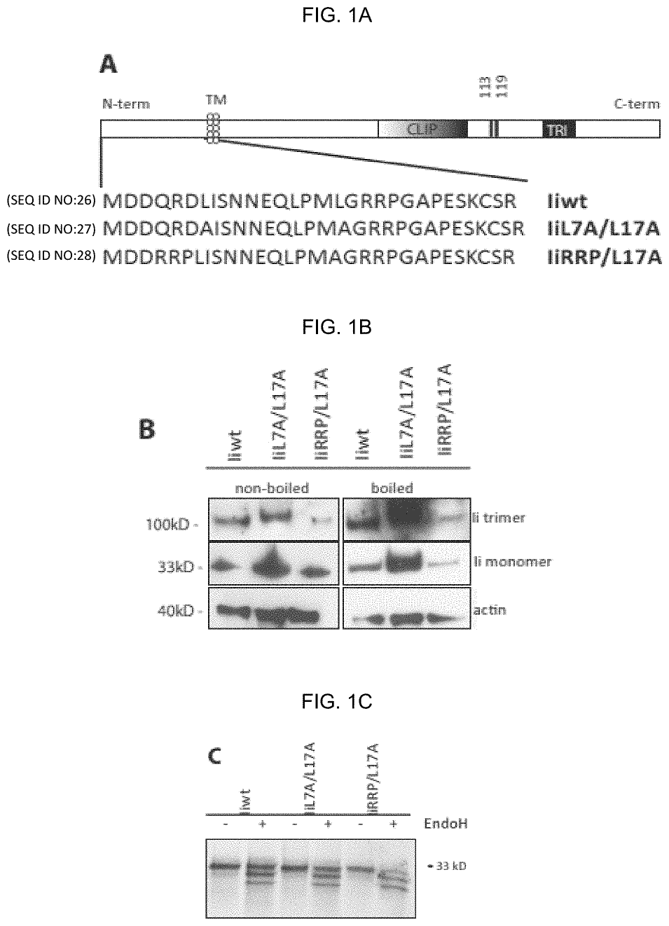

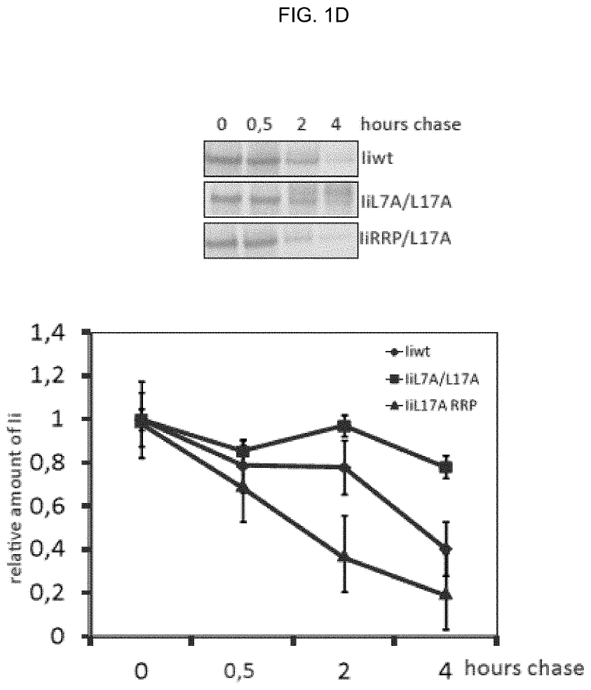

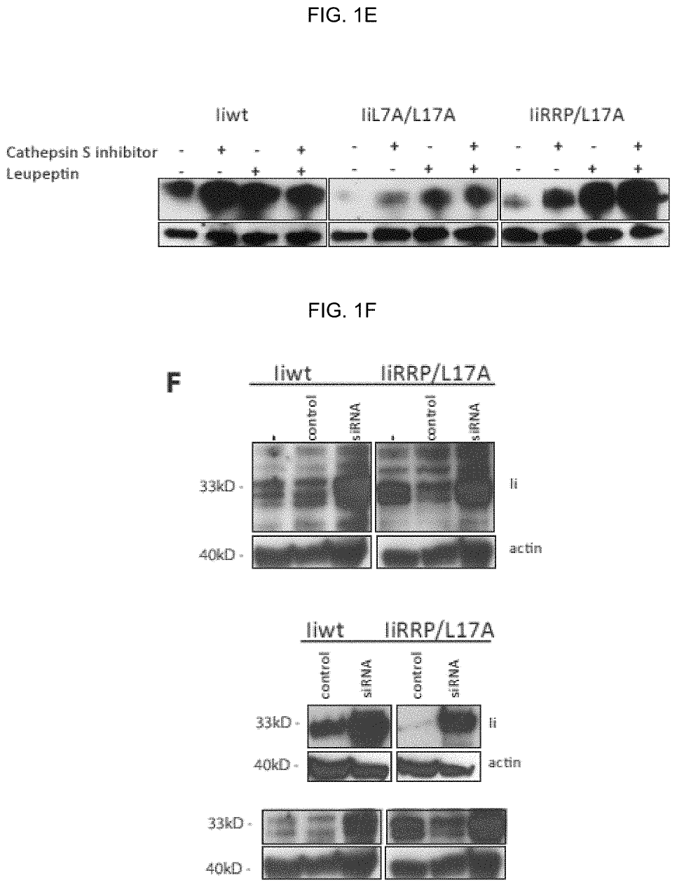

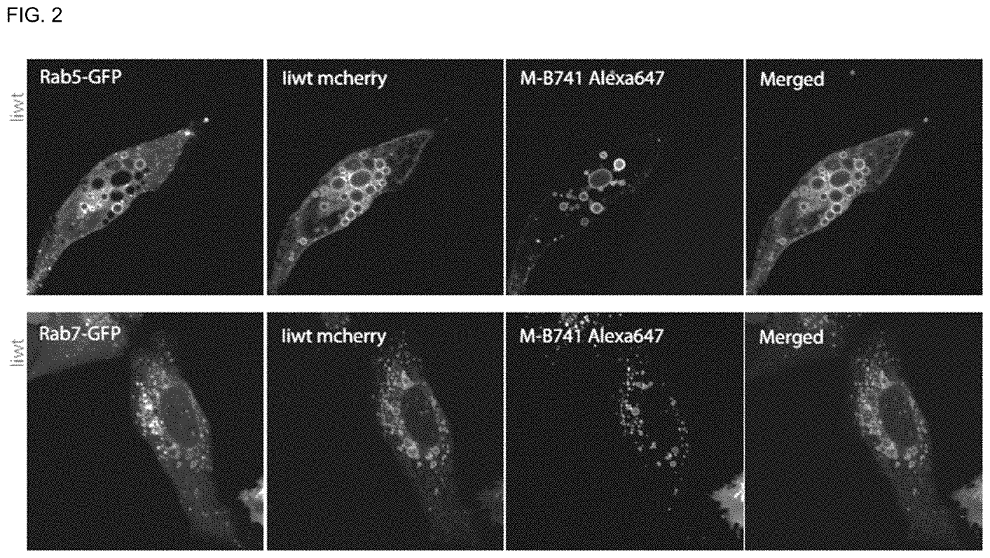

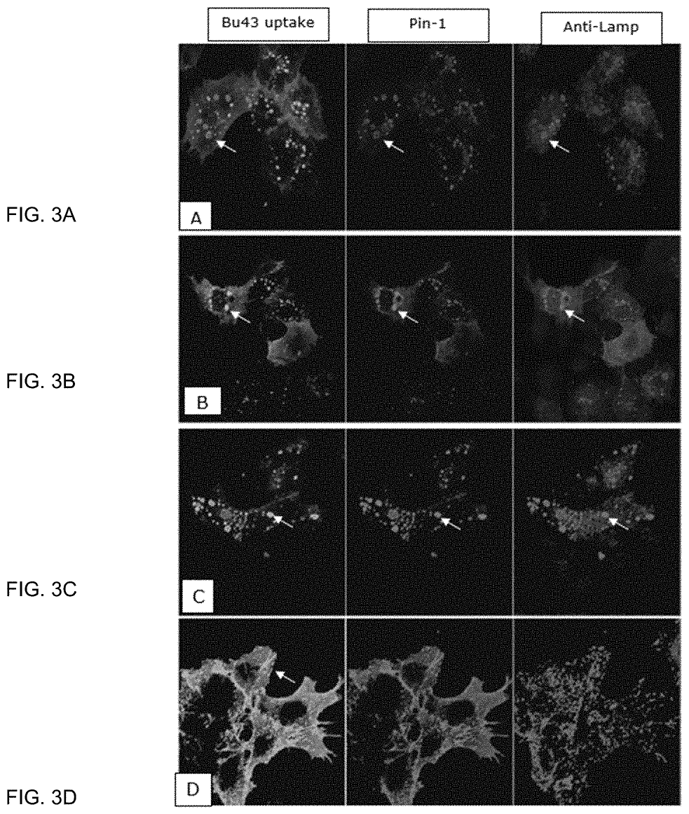

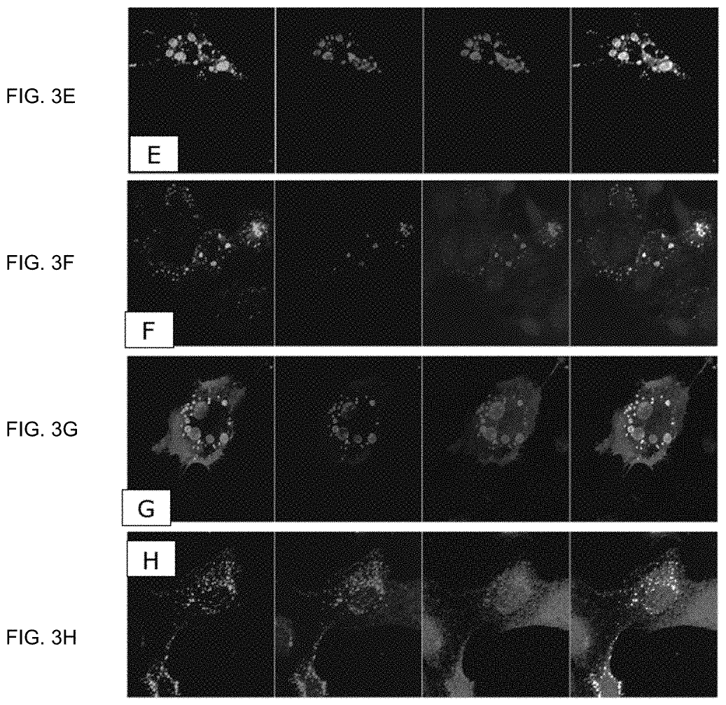

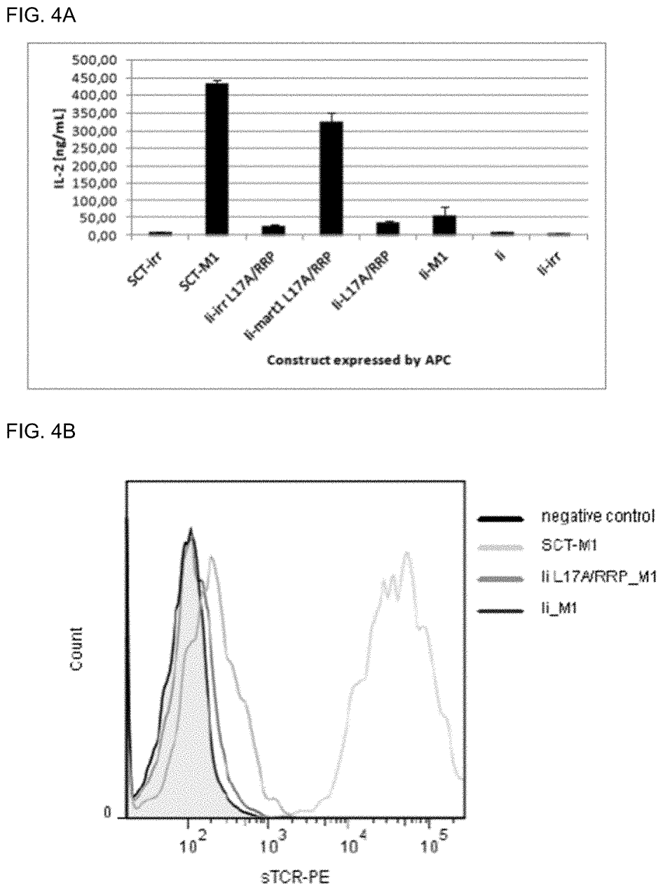

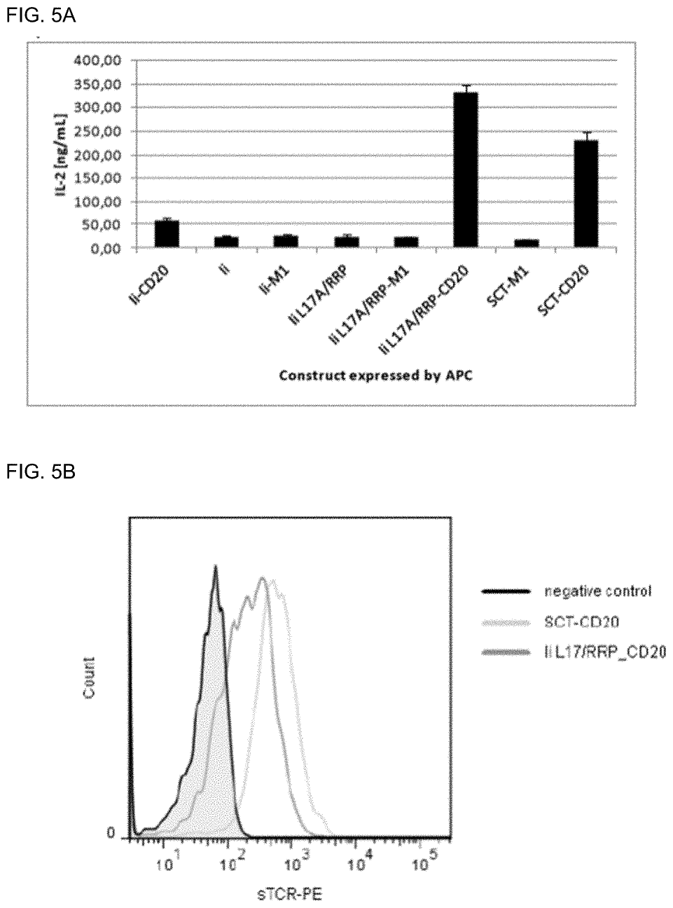

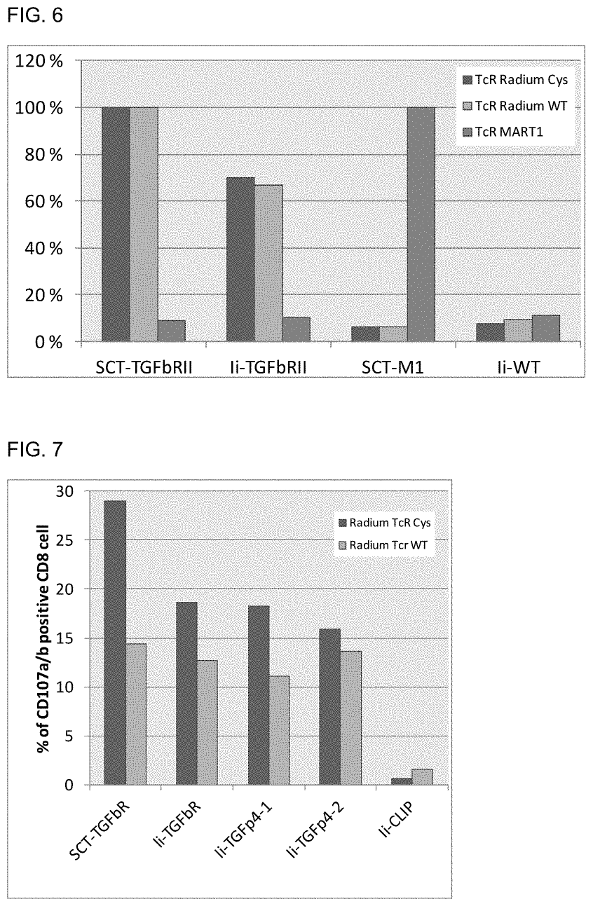

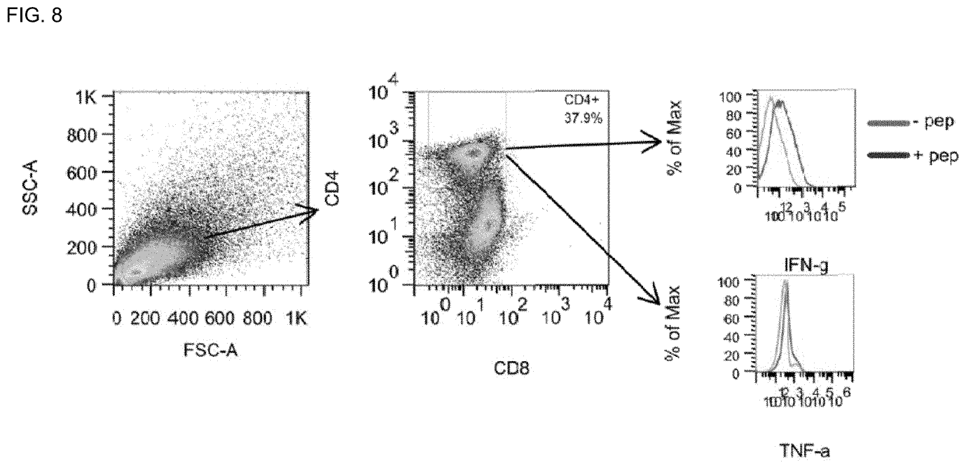

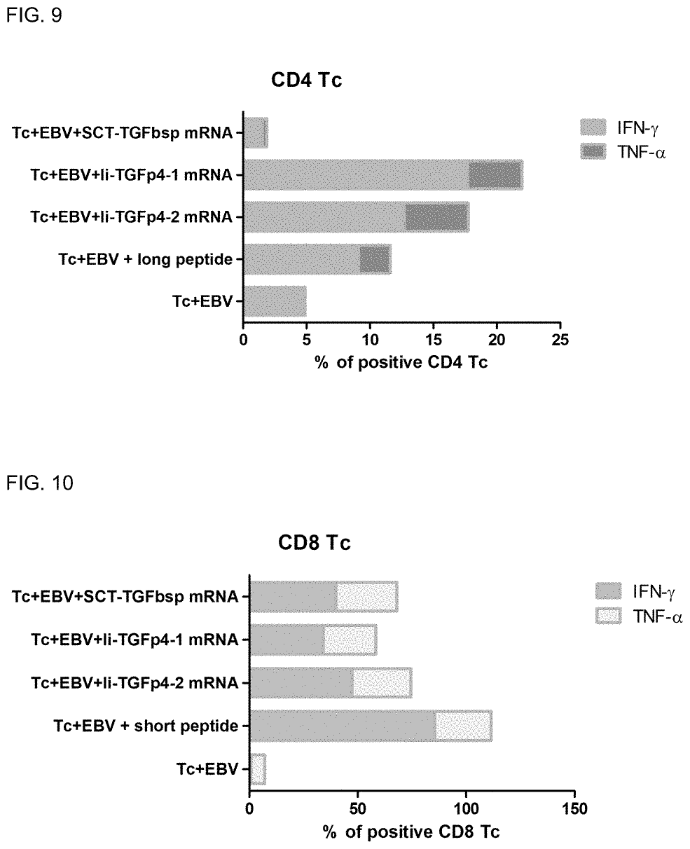

FIG. 1D

Hek cells were transfected as indicated. The cells were pulsed with 35SMethionine/Cystein containing media for 30 min, washed and chased for the indicated timepoints. Ii was immunoprecipitated with anti Ii M-B741, (while 10% of the lysates were subjected to western blotting). Iiwt has a half life of about 3 hours. The double mutant, IiL7AL17A, as it is unable to internalize, slowly continues to accumulate in the cell and would need longer chase timepoints for the half life to be determined. IiRRP/L17A, however, shows a half life of approximately 1 hour supporting a faster kinetic to endosomal compartments.

FIG. 1E

Transfected Hek cells were subjected to a metabolic pulse with 35SMethionine/Cystein containing media for 30 min. During this, the Cathepsin S inhibitor and Leupeptin were added either alone or in combination. Cells were lysed and Ii was immunoprecipitated with anti Ii M-B741, while 10% of the lysates were used for WB. Ii RRP/L17A displayed the strongest protection from degradation.

FIG. 1F

HeLa cells were treated with siRNA as described in material and methods. 72 h post treatment, cells were transfected with Iiwt and IiRRP/L17A. AP3 depletion resulted in accumulation of both Iiwt and Ii RRP/L17A, however, IiLRRP/L17A was protected against degradation to a higher extent than Iiwt, suggesting that the AP3 depletion interfered with trafficking of this Ii mutant.

FIG. 2 shows MDCK cells with indicated fluorescence.

FIG. 3A-H shows cellular Distribution of Ii wt and Ii Mutants. M1 cells were seeded on coverslips and transiently transfected with Ii wt and mutants as indicated in the figure. Green channel; Ii luminal domain, red channel; Ii cytoplasmic tail, blue channel; Lamp-1. Arrows indicate endosomes positive for the different stainings. Bar 40 μm. The Ii wt (A), Ii L7A (B) and the Ii L17A (C) constructs were located at the plasma membrane and throughout the endosomal pathway, supporting that one intact leucine signal is sufficient for Ii sorting. However Ii L7A/L17A, was permanently located at the plasma membrane unable to internalize as the sorting signals were removed (D). Ii L7A RAP (E), Ii L7A RRP (F) and Ii RAP L17A (G) all had internalized Bu43, indicating that they reached the cell surface. They were also found in endosomes positive for Lamp-1. Ii L17A RAP (H), however, had endosomes devoid of Bu43 uptake. Cells expressing this protein had highly reduced Bu43 labeling at the cell surface, but Ii cytoplasmic tails were found to co-localize with Lamp-1. Co-localization between Pin-1 and Lamp-1 devoid of Bu43 strongly indicated that a fraction of Ii L17A RRP sorted directly to Lamp-1 positive compartments without ever reaching the cell surface.

FIG. 4A-B

L17A/RRP mutant increased presentation of antigenic peptide on MHC-I. (A) J76 cells constitutively expressing DMF5 were incubated for 12 hours with SupT1 (for SCT) or SupT1(HLA-A2 positive) transduced with the indicated constructs. Supernatants were harvested and IL-2 was detected by ELISA. A representative experiment of 2 is shown. Bars are average value+/−SD from duplicate. Irr=control peptide (B) SupT1 (for SCT) or SupT1(HLA-A2 positive) transduced with the indicated construct were incubated with sTcR (10 nM final concentration) for 15 minutes at RT and the bound complex was detected using anti-His-PE. This experiment was performed twice.

FIG. 5A-B same as in FIG. 3 but with CD20p replacing CLIP peptide and CD20 TcR expressing J76 cells. (A) J76 expressing a CD20-specific TCR were incubated with presenting cells expressing the indicated constructs (Ii+HLA-A2) or SCT. After 2 hours the supernatants were harvested and IL-2 was detected by ELISA. Columns are duplicates and error bars=SD. A representative experiment of two is shown here. (B) SupT1, SCT-CD20 or Ii-CD20+HLA-A2 transduced SupT1 cells were incubated with 10 nM CD20 specific sTCR for 15 minutes at RT and the bound complex was detected using anti-His-PE. This experiment was performed twice.

FIG. 6 shows APC expressing the indicated constructs were incubated with T cells expressing TCR Radium 1 (TGFbRII-specific) or DMF5 (MART1 specific). IL-2 release was used to monitore Tc activation and plotted as % of max, which is activation when incubated with single chain trimer.

FIG. 7 shows same as FIG. 6, but the readout is CD107a expression. Here Ii expressing long peptides (p4-1 and p4-2) were compared to Ii-TGFbRII or SCT-TGFbRII. Ii-wild type (Ii-CLIP) was used as a negative control.

FIG. 8 shows Top: gating strategy, here CD4 Tc were checked for IFN-g (top) and TNF-α (bottom) expression upon specific stimulation. Presenting cells were autologous EBV-B cells electroporated with the indicated mRNA, CD4 Tc: quantification of the % of cells positive for either or both IFN-g and TNF-α. CD8 Tc: same as for CD4.

FIG. 9 shows CD4+ T-cells and INF-gamma versus TNF-alpha response.

FIG. 10 shows CD8+ T-cells and INF-gamma versus TNF-alpha response.

### DETAILED DESCRIPTION OF THE INVENTION

The present invention relates to the effect on MHC I antigen presentation with Ii trafficking mutants.

In one embodiment of the present invention is the Ii trafficking mutants capable of CD8⁺ and/or CD4⁺ T-cell activation.

In another embodiment of the present invention, the mutants were designed to bind AP-3 rather than AP-1 and -2.

In this way the present inventors ensured that Ii is sorted rapidly and most likely directly to the endosomal pathway. Indeed, the mutant IiRRP/L17A was directly transported to the late endosomes and therefore had a shorter half-life compared to Iiwt.

More so, Ii RRP/L17A where CLIP was exchanged with tumor derived peptides, was found to be extremely potent in CD8+ T cell activation, and was 3-6 times more potent than its Iiwt counterpart (see examples).

Taken together, these data support an Ii-based loading of MHC I.

The present inventors have shown that the process can be further improved when the targeting of Ii is modified, suggesting that direct targeting to late endosomes represent an ideal source of MHC-I loading material.

Thus, an object of the present invention is to provide molecules that present specified antigens in an MHC class I context on the surface of a cell to generate CD8⁺ T-cell activation.

An aspect of the present application relates to a nucleic acid molecule encoding a type II transmembrane invariant chain (Ii) which is modified by exchanging the class II-associated Ii peptide (CLIP) with an antigen.

In one embodiment of the present invention is one or more sorting motifs is/are replaced with a AP-3 binding motif.

In another embodiment of the present invention is the nucleic acid molecule selected from the group consisting of mRNA and DNA.

In another embodiment of the present invention is the nucleic acid molecule mRNA.

Another aspect of the present application relates to an amino acid molecule comprising the type II transmembrane invariant chain (Ii) according to the present invention.

In one embodiment of the present invention, the antigen also activates a CD4⁺ T-cell.

In another embodiment of the present invention, the antigen activates both CD4+ and CD8+ T cells.

In a further embodiment of the present invention, the antigen is comprised of multiple epitopes for the stimulation of both CD4+ and CD8+ T cells. These epitopes can stimulate CD4+ and CD8+ cells separately.

In another embodiment of the present invention, is the T cell stimulation not restricted to a specific HLA genotype.

The present invention also relates to a combination of known antigens with the known CLIP substitution to generate a new constructs used for DC vaccination for colorectal cancer patients with microsatellite instability (MSI).

In addition, the inventors show presentation to and activation of both CD4+ and CD8+ T-cells using the long peptides described below (TGFp4-1 and TGFp4-2). This is new and not obvious from the prior art as prior art has focused on CD8+ responses. The experiments are show in examples 1, 2, and 3, and FIGS. 7-11.

### Sorting Motif

AP3 binds both leucine ([DE]xxxL[LI]) and tyrosine (YxxØ) based sorting motifs through their β- and μ-chain respectively (Craig H M Virology 2000), Dell' Angelica EC EMBO J 1997).

Thus in one embodiment of the present invention is the sorting motif a leucine ([DE]xxxL[LI]) or a tyrosine (YxxØ) based sorting motif.

The residues N terminal to the two (iso)leucines of the sorting signal determine adaptor binding (Rodionov 2002) and certain residues favor AP3 binding. LIMPII is a well-known AP3 binder harboring a RAP motif in front of the LI signal.

An embodiment of the present invention it thus where a RAP motif is placed in front of the LI signal.

AP3 is required for mouse CD1d (RRRSAYQDIR (SEQ ID NO:29)) mediated antigen presentation of glycosphingolipids to NKT cells (Elewaut D 2003, JEM and Lawton A P, JI 2005). Human CD1b (RRRSYQNIP (SEQ ID NO:30)) is the only human CD molecule being dependent on AP3 for proper trafficking and antigen presentation (Sugita M, Immunity 2002).

These molecules harbor tyrosin based sorting motifs, however the requirement for positively charged Arginine residues seems to be similar to both leucine—and tyrosine based sorting signals.

Upstream residues are important for AP3 binding to tyrosin signals.

In one embodiment of the present invention are the upstream residues optimized for AP-3 binding.

In another embodiment can Lysines replace Arginines.

In one embodiment of the present invention is the sorting motif is a leucine or a tyrosine based sorting motif

In a further embodiment is the mutant L17A and the upstream region has been optimised wherein QRD has been changed to RAP, RRP, QAP, RAD, QRP, RAD, QRP, QAD, or RRD.

In another embodiment of the mutant L7A and the upstream region has been optimised wherein QLP has been changed to RAP, RLP, QAP, or RRP.

In a further embodiment is the upstream region optimised wherein NEQLP has been replaced with DERAP (SEQ ID NO:31).

In another embodiment of the present invention is the one or more sorting signals is/are a cytoplasmic sorting motif selected from the group consisting of Leu₇/Ile₈ and Met₁₆/Leu₁₇.

In a further embodiment of the present invention is the sorting motif (QLP)L7I (SEQ ID NO:32) is replaced with (RRP)L7I (SEQ ID NO:33) and/or (QRD)L17A (SEQ ID NO:34) is replaced with (RRP)L17A (SEQ ID NO:35).

### Sequences and Antigens

The protein sequence of Iiwt (SEQ ID NO: 1) is:

MDDQRDLISNNEQLPMLGRRPGAPESKCSRGALYTGFSILVTLLLAGQAT
TAYFLYQQQGRLDKLTVTSQNLQLENLRMKLPKPPKPVSKMRMATPLLMQ
ALPMGALPQGPMQNATKYGNMTEDHVMHLLQNADPLKVYPPLKGSFPENL
RHLKNTMETIDWKVFESWMHHWLLFEMSRHSLEQKPTDAPPKESLELEDP
SSGLGVTKQDLGPVPM

CLIP is the underlined sequence above (SEQ ID NO: 2):

MRMATPLLM

Iiwt-TGFbRIIp is Iiwt wherein CLIP has been changed to TGFbRIIp (SEQ ID NO: 3):

MDDQRDLISNNEQLPMLGRRPGAPESKCSRGALYTGFSILVTLLLAGQAT
TAYFLYQQQGRLDKLTVTSQNLQLENLRMKLPKPPKPVSKRLSSCVPVAQ
ALPMGALPQGPMQNATKYGNMTEDHVMHLLQNADPLKVYPPLKGSFPENL
RHLKNTMETIDWKVFESWMHHWLLFEMSRHSLEQKPTDAPPKESLELEDP
SSGLGVTKQDLGPVPM

TGFbRIIp is underlined in the above sequence (SEQ ID NO: 4):

RLSSCVPVA

Iimut-TGFbRIIp (SEQ ID NO: 5) is (SEQ ID NO: 3) three mutations have been introduced (underlined):

MDDRRPLISNNEQLPMAGRRPGAPESKCSRGALYTGFSILVTLLLAGQAT
TAYFLYQQQGRLDKLTVTSQNLQLENLRMKLPKPPKPVSKRLSSCVPVAQ
ALPMGALPQGPMQNATKYGNMTEDHVMHLLQNADPLKVYPPLKGSFPENL
RHLKNTMETIDWKVFESWMHHWLLFEMSRHSLEQKPTDAPPKESLELEDP
SSGLGVTKQDLGPVPM

The constructs used in examples 2 and 3 are:

(SEQ ID NO: 3)

MDDQRDLISNNEQLPMLGRRPGAPESKCSRGALYTGFSILVTLLLAGQAT
TAYFLYQQQGRLDKLTVTSQNLQLENLRMKLPKPPKPVSKRLSSCVPVAQ
ALPMGALPQGPMQNATKYGNMTEDHVMHLLQNADPLKVYPPLKGSFPENL
RHLKNTMETIDWKVFESWMHHWLLFEMSRHSLEQKPTDAPPKESLELEDP
SSGLGVTKQDLGPVPM

TGFbRIIp is underlined in the above sequence:

(SEQ ID NO: 4)

RLSSCVPVA

(SEQ ID NO: 6)

MDDQRDLISNNEQLPMLGRRPGAPESKCSRGALYTGFSILVTLLLAGQAT
TAYFLYQQQGRLDKLTVTSQNLQLENLRMKLPKPPKPVSKKSLVRLSSCV
PVALMSAMTQALPMGALPQGPMQNATKYGNMTEDHVMHLLQNADPLKVYP
PLKGSFPENLRHLKNTMETIDWKVFESWMHHWLLFEMSRHSLEQKPTDAP
PKESLELEDPSSGLGVTKQDLGPVPM

TGFbRIIp4-1 is underlined in the above sequence:

(SEQ ID NO: 7)

KSLVRLSSCVPVALMSAMT

(SEQ ID NO: 8)

MDDQRDLISNNEQLPMLGRRPGAPESKCSRGALYTGFSILVTLLLAGQAT
TAYFLYQQQGRLDKLTVTSQNLQLENLRMKLPKPPKPVSKSLVRLSSCVP
VALMSAMTTSSSQQALPMGALPQGPMQNATKYGNMTEDHVMHLLQNADPL
KVYPPLKGSFPENLRHLKNTMETIDWKVFESWMHHWLLFEMSRHSLEQKP
TDAPPKESLELEDPSSGLGVTKQDLGPVPM

TGFbRIIp4-2 is underlined in the above sequence:

(SEQ ID NO: 9)

SLVRLSSCVPVALMSAMTTSSQ

Ii L7A is

(SEQ ID NO 20)

MDDQRDAISNNEQLPMLGRRPGAPESKCSR.

Ii L17A is

(SEQ ID NO 21)

MDDQRDLISNNEQLPMAGRRPGAPESKCSR.

Ii L7A/L17A is

(SEQ ID NO 22)

MDDQRDAISNNEQLPMAGRRPGAPESKCSR.

Ii L7A RAP is

(SEQ ID NO 23)

MDDQRDAISNNERAPMLGRRPGAPESKCSR.

### Ii L7a RRP is

(SEQ ID NO 24)

MDDQRDAISNNERRPMLGRRPGAPESKCSR.

Ii L17A RAP is

(SEQ ID NO 25)

MDDRAPLISNNEQLPMLGRRPGAPESKCSR.

One aspect of the present invention relates to the sequences listed above in any of the contexts mentioned herein.

Thus, the constructs can be the exact construct listed or they can contain variations.

Other peptides or antigens of interest may be inserted into Iiwt by exchanging CLIP with a peptide or antigen of interest.

Another aspect of the present invention relates to an isolated amino acid molecule that has an open reading frame (ORF) amino acid sequence with 80% sequence identity to the sequences of the present invention, such as 90%, such as 95%, such as 98%, such as 99%.

As commonly defined “identity” is here defined as sequence identity between genes or proteins at the nucleotide or amino acid level, respectively.

Thus, in the present context “sequence identity” is a measure of identity between proteins at the amino acid level and a measure of identity between nucleic acids at nucleotide level. The protein sequence identity may be determined by comparing the amino acid sequence in a given position in each sequence when the sequences are aligned. Similarly, the nucleic acid sequence identity may be determined by comparing the nucleotide sequence in a given position in each sequence when the sequences are aligned.

To determine the percent identity of two amino acid sequences or of two nucleic acids, the sequences are aligned for optimal comparison purposes (e.g., gaps may be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100).

In one embodiment the two sequences are the same length.

In another embodiment the two sequences are of different length and gaps are seen as different positions.

One may manually align the sequences and count the number of identical amino acids. Alternatively, alignment of two sequences for the determination of percent identity may be accomplished using a mathematical algorithm. Such an algorithm is incorporated into the NBLAST and XBLAST programs of (Altschul et al. 1990). BLAST nucleotide searches may be performed with the NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention. To obtain gapped alignments for comparison purposes, Gapped BLAST may be utilised. Alternatively, PSI-Blast may be used to perform an iterated search which detects distant relationships between molecules. When utilising the NBLAST, XBLAST, and Gapped BLAST programs, the default parameters of the respective programs may be used. See the world wide web at ncbi.nlm.nih.gov. Alternatively, sequence identity may be calculated after the sequences have been aligned e.g. by the BLAST program in the EMBL database (See the world wide web at ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous. NBLAST program, score=100, wordlength=12, to obtain nucleotide sequences homologous to a nucleic acid molecules of the invention. BLAST protein searches may be performed with the XBLAST program, score=50, wordlength=3 to obtain amino acid sequences homologous to a protein molecule of the invention. To obtain gapped alignments for comparison purposes, Gapped BLAST may be utilised. Alternatively, PSI-Blast may be used to perform an iterated search which detects distant relationships between molecules. When utilising the NBLAST, XBLAST, and Gapped BLAST programs, the default parameters of the respective programs may be used. See the world wide web at ncbi.nlm.nih.gov. Alternatively, sequence identity may be calculated after the sequences have been aligned e.g. by the BLAST program in the EMBL database (See the world wide web at ncbi.nlm.gov/cgi-bin/BLAST). Generally, the default settings with respect to e.g. “scoring matrix” and “gap penalty” may be used for alignment. In the context of the present invention, the BLASTN and PSI BLAST default settings may be advantageous.

The percent identity between two sequences may be determined using techniques similar to those described above, with or without allowing gaps. In calculating percent identity, only exact matches are counted.

An embodiment of the present invention thus relates to sequences of the present invention that have some degree of sequence variation.

The variation is typically achieved through conservative or sense mutations which will allow the retention of functionality.

Thus, one embodiment of the present invention relates to the sequences disclosed herein, in which 50, such as 30, such as 20, such as 15, such as 10, such as 8, such as 5, such as 4, such as 3, such as 2, such as 1 amino or nucleic acid has been exchanged. Preferably through a conservative or sense mutation.

It should be noted that while several of the sequences in the present application are DNA sequences, the present invention contemplates the corresponding RNA sequence, and DNA and RNA complementary sequences as well.

Thus, in cases where a DNA sequence is mentioned refers such DNA sequence also to the RNA equivalent i.e. with Ts exchanged with Us as well as their complimentary sequences.

In another embodiment, the nucleic acid further comprises a reporter gene, which, in one embodiment, is a gene encoding neomycin phosphotransferase, Renilla luciferase, secreted alkaline phosphatase (SEAP), Gaussia luciferase or fluorescent proteins such as green or red fluorescent protein. Reporter genes can for example be beneficial in tracking intracellular movements, antigen presentation or transfection efficiency.

CLIP can be exchanged with all conceivable antigens.

In one embodiment is CLIP exchanged with CD20p

(SEQ ID NO: 6):

SLFLGILSV

In one embodiment is CLIP exchanged with MART1p:

(SEQ ID NO: 7)

AAGIGILTV:
or

(SEQ ID NO: 8)

ALGIGILTV.

An antigen is any substance which provokes an adaptive immune response. An antigen is often foreign or toxic to the body (for example, a bacterium or virus) which, once in the body, attracts and is bound to a respective and specific antibody.

Cells present their antigenic structures to the immune system via a histocompatibility molecule. Depending on the antigen presented and the type of the histocompatibility molecule, several types of immune cells can become activated.

Antigen was originally a structural molecule that binds specifically to the antibody, but the term now also refers to any molecule or molecular fragment that can be recognized by highly variable antigen receptors (B-cell receptor or T-cell receptor) of the adaptive immune system.

For T-Cell Receptor (TCR) recognition, it must be processed into small fragments inside the cell and presented to a T-cell receptor by major histocompatibility complex (MHC).

In one embodiment of the present invention is the antigen a CD4⁺ T-cell activating antigen and/or a CD8⁺ T-cell activating antigen.

In yet another embodiment of the present invention is wherein the antigen is a tumor or a viral antigen.

A tumor is an abnormal mass of tissue as a result of abnormal growth or division of cells. Prior to abnormal growth (known as neoplasia), cells often undergo an abnormal pattern of growth, such as metaplasia or dysplasia. However, metaplasia or dysplasia do not always progress to neoplasia.

The growth of neoplastic cells exceeds, and is not coordinated with, that of the normal tissues around it.

The growth persists in the same excessive manner even after cessation of the stimuli. It usually causes a lump or tumor. Neoplasms may be benign, pre-malignant (carcinoma in situ) or malignant (cancer).

Benign neoplasms include uterine fibroids and melanocytic nevi (skin moles). They are circumscribed and localized and do not transform into cancer.

Potentially malignant neoplasms include carcinoma in situ. They do not invade and destroy but, given enough time, will transform into a cancer.

Malignant neoplasms are commonly called cancer. They invade and destroy the surrounding tissue, may form metastases and eventually kill the host.

Secondary neoplasm refers to any of a class of cancerous tumor that is either a metastatic offshoot of a primary tumor, or an apparently unrelated tumor that increases in frequency following certain cancer treatments such as chemotherapy or radiotherapy.

The antigen can also originate from a bacteria or a virus.

It will be beneficial if such an antigen can be presented to generate both CD4⁺ and CD8⁺ T-cell activation.

Another aspect of the present application relates to a pharmaceutical composition comprising the nucleic acid molecule or the amino acid molecule according to the present invention, and a pharmaceutically acceptable carrier, excipient and/or diluent.

In one embodiment of the present invention is the pharmaceutical composition a vaccine.

In another embodiment, this invention provides for compositions comprising an isolated nucleic acid, vector or cell of this invention, or an isolated nucleic acid obtained via the methods of this invention.

In one embodiment, the term “composition” refers to any such composition suitable for administration to a subject, and such compositions may comprise a pharmaceutically acceptable carrier or diluent, for any of the indications or modes of administration as described.

The active materials in the compositions of this invention can be administered by any appropriate route, for example, orally, parenterally, intravenously, intradermally, subcutaneously, or topically, in liquid or solid form.

It is to be understood that any applicable drug delivery system may be used with the compositions and/or agents/vectors/cells/nucleic acids of this invention, for administration to a subject, and is to be considered as part of this invention.

Design of such administration and formulation is routine optimization generally carried out without difficulty by the practitioner.

It is to be understood that any of the methods of this invention, whereby a nucleic acid, vector or cell of this invention is used, may also employ a composition comprising the same as herein described, and is to be considered as part of this invention.

“Pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopoeia for use in animals, and more particularly in humans.

The term “excipient” refers to a diluent, adjuvant, carrier, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

The term “adjuvant” refers to a compound or mixture that enhances the immune response to an antigen. An adjuvant can serve as a tissue depot that slowly releases the antigen and also as a lymphoid system activator that non-specifically enhances the immune response. Often, a primary challenge with an antigen alone, in the absence of an adjuvant, will fail to elicit a humoral or cellular immune response.

Adjuvants include, but are not limited to, complete Freund's adjuvant, incomplete Freund's adjuvant, saponin, mineral gels such as aluminum hydroxide, surface active substances such as lysolecithin, pluronicpolyols, polyanions, peptides, oil or hydrocarbon emulsions, keyhole limpet hemocyanins, dinitrophenol, and potentially useful human adjuvants such as BCG (bacilleCalmette-Guerin), Corynebacteriumparvmm, aluminum hydroxide+MPL, Addavax, MF59, CAF01, CAF04, CAF05 and CAF09, and Sigma adjuvant system.

Preferably, the adjuvant is pharmaceutically acceptable.

### Methods

Another aspect of the present application relates to a method of presenting a CD8⁺ T-cell activating antigen on a cell, comprising modifying the type II transmembrane invariant chain (Ii) by exchanging the class II-associated Ii peptide (CLIP) with an antigen, replacing one or more sorting motifs with a AP-3 binding motif, and introducing the Ii to a cell.

This method will allow CD8⁺ and optionally CD4⁺ T-cell activation to the antigen inserted instead of CLIP.

Another aspect of the present application relates to a method for inducing a CD8⁺ and/or a CD4⁺ response, comprising administering the nucleic acid molecule or the amino acid molecule according to the present invention to an individual.

In one embodiment of the present invention is the nucleic acid molecule or amino acid molecule administered using electroporation or transfection. Other means of administration are listed above.

Another aspect of the present application relates to the nucleic acid molecule or the amino acid molecule according to the present invention for use as a medicament.

A further aspect of the present application relates to a nucleic acid molecule or the amino acid molecule of the present invention for use in the treatment of a disease that is associated with the antigen.

### EXAMPLES

### Example 1—Sorting of an Engineered Invariant Chain Molecule Directly to Endosomal Pathway Leads to Improved MHC Class I Loading

### Introduction

CD4⁺ T cells recognize exogenously derived peptides presented on the cell surface of antigen presenting cells in the MHC class II (MHC II) context. The biosynthesis and transport of MHC II molecules are tightly regulated by the type II transmembrane invariant chain (Ii). Ii harbors two leucine based sorting signals; Leu7/Ile8 and Met16/Leu17 in its cytoplasmic tail. Indeed, these signals direct the Ii-MHC II complex to the endosomal pathway mainly via the cell surface, (therefore called the indirect pathway) and through binding to the adaptor proteins AP-1 and AP-2. The APs are involved in the formation of clathrin-coated vesicles at the trans Golgi and plasma membrane respectively. In addition, they play a pivotal role in cargo selection by recognizing the appropriate sorting signals of integral membrane proteins. Either one of the two Ii leucine signals is sufficient for targeting Ii to endosomal compartments, but the Leu7/Ile8 is more potent in doing so.

Within the endosomes, Ii is sequentially degraded leaving the class II-associated Ii peptide (CLIP) bound to the MHC II groove. CLIP is subsequently exchanged for antigenic peptides prior to transport to the cell surface for presentation. Several studies have shown that by genetically exchanging the CLIP region with antigenic peptides, MHC II molecules are efficiently loaded with the peptide and presented to specific CD4+ T cells.

Unlike MHC II, MHC I binds mainly endogenously derived peptides generated by the proteasome in the cytosol which are targeted to the endoplasmic reticulum (ER) via TAP transporters. After MHC I peptide loading, the complex is presented to cytotoxic CD8+ T cells. MHC I is independent of Ii, however, it was recently demonstrated that Ii plays a physiological role for targeting MHC I to the endosomal pathway for loading of viral peptides and cross-presentation to CD8+ T cells. Additional evidence for an Ii-MHC I interaction came from van Luijn and colleagues who showed that CLIP efficiently binds to several MHC I molecules in leukemic cells. Furthermore, we recently showed that when CLIP is exchanged with an MHC I tumor specific antigen, the peptide loads onto MHC I in a proteasome/TAP/tapasin independent manner. This strategy was found to be as efficient as exogenous loading of synthetic peptide in vitro. In conclusion, the data clearly corroborates an Ii influence on not only MHC II, but also on MHC I antigen presentation.

In this study we investigated the effect on MHC I antigen presentation with Ii trafficking mutants which were designed to bind AP-3 rather than AP-1 and -2. In this way we ensured that Ii sorted rapidly and directly to the endosomal pathway. Indeed, the mutant IiRRP/L17A was directly transported to the late endosomes and therefore had a shorter half-life compared to Iiwt. More so, Ii RRP/L17A where CLIP was exchanged with tumor derived peptides, was found to be extremely potent in CD8+ T cell activation, and was 3-6 times more potent than its Iiwt counterpart. Taken together, these data support an Ii-based loading of MHC I. We show that the process can be further improved when the targeting of Ii is modified, suggesting that direct targeting to late endosomes represent an ideal source of MHC-I loading material.

### Methods

Recombinant cDNA Constructs

cDNA encoding human Iip33 wt, was subcloned into the pcDNA3 expression vector at KpnI-BamHI. Human Iip33 mutants; Ii L17A and Ii L7A L17A in the PSV51L expression vector have also been described in Simonsen et al. (International Immunology 1993)KpnI and BamHI restriction sites were introduced up- and downstream of the Ii sequences respectively, by PCR. The Ii mutants were thereafter subcloned into pcDNA3 at KpnI-BamHI, behind the T7-RNA polymerase promoter.

Ii constructs described above were used as templates for PCR quick change mutagenesis (all reagents used were included in the kit; QuickChange® Site-Directed Mutagenesis (Stratagen, La Jolla, Calif., USA)) in order to generate the AP-3 binding motif RRP.

| Primer name | Primer sequence |
|---|---|
| Ii KpnI forward | 5′ AGAGA GGGTACCGTCATGGATGAC |
| | CAGCGCGAC (SEQ ID NO: 10) |
| Ii BamHI reverse | 5′ AGAGAGGGATCCTCACATGGGG |
| | ACTGGGCCCAG (SEQ ID NO: 11) |
| L17A RRP sense | 5′-CCGTCATGGATGACCGTCGTCCCCTTA |
| | TCTCCAACAATG-3′ (SEQ ID NO: 12) |
| L17A RRP anti-sense | 5′- |
| | CATTGTTGGAGATAAGGGGACGACGGTCATC |
| | CATGACGG-3′ (SEQ ID NO: 13) |

*Point mutations are underlined

All CLIP-antigenic peptide constructs (IiMART1 and IiCD20) were cloned by site direct mutageneis of the Iiwt and IiRRP/L17A construct subcloned in pENTR vector (Invitrogen, Oslo, Norway). The mutagenesis to change the CLIP peptide (MRMATPLLM (SEQ ID NO:36)) into antigenic peptides (CD20: SLFLGILSV (SEQ ID NO:37) and MART1: ELAGIGILTV (SEQ ID NO:38)) was performed with IiMART1 forw/IiMART1 rev primers for IiMART1 and IiCD20 forw/IiCD20 rev for IiCD20 (table 1). After sequence verification these constructs were recombined into a Gateway-converted pCI-pA102 (Walchli et al. 2011).

### Cell Lines

M1 cells, HEK293 cells, human epithelial HeLa-Kyoto and Madin Darby Canine Kidney (MDCK) cells were grown in Dulbecco's Modified Eagle Medium (DMEM, Bio Witthaker, Walkersville, Md., USA). All medias were supplemented with heat-inactivated 10% fetal calf serum (FCS, HyClone, Logan, Utah, USA). J76 were a kind gift from Miriam Hemskerk (Leiden University Medical Center, The Nederland), SupT1 from Martin Pule (University College London, UK), both cell lines were grown in RPMI+10% fetal calf serum. PBMC from healthy donor etc.

### Antibodies and Reagents

The Bu43 antibody, was kindly provided by D. Harding (Birmingham, UK). M-B741 was purchased from BD Biosciences (Franklin Lakes, N.J., USA). Pin-1, anti-Lamp-1 and anti-actin were all purchased from AbCam, (Cambridge, UK).

The secondary antibodies; anti murine IgM FITC, goat anti-rabbit alexa 647, goat anti-mouse alexa 555, sheep anti-mouse-HRP, were all aquired from Invitrogen/Bio-Rad (Hercules, Calif., USA). FITC mouse anti-rat IgG2b and Rat IgG2b, were purchased from BD Biosciences.

### Biochemical Analyses

Metabolic labeling was done using ³⁵S-labeled Cystein/Methionine (Perkin Elmer, Waltham, Mass., USA). Cells were seeded to 60%-70% confluence; washed three times in Cys/Met-free DMEM; incubated in Cys/Met-free DMEM for 45 min followed by a 30 min pulse with Cys/Met-free DMEM supplemented with 50 μCi S35. For the pulse chase assay the cells were washed three times in DMEM containing 2 mM L-glutamine, primocin, and 30% FCS and chased for indicated time periods. Immunoprecipitations were done at at 4° C. over night with 1-2 μg ml-l antibody in lysis buffer (50 mM Tris-HCl, pH 7.5, 150 mM NaCl, 1% Tx100) supplemented with the protease inhibitor coctail Protease Arrest (GBiosciences, St. Louis, Mo., USA). Antigen-antibody complexes were captured with Protein G-coupled DYNABEADS' reagent (Invitrogen). For the experiments including protease inhibitors, the same procedure was followed as for metabolic labeling. During the 30 min S35-Cys/Met pulse, 20 nM Cathepsin S Inhibitor (Merck Chemicals Ltd., Nottingham, UK) and/or 100 μM Leupeptin (SIGMA ALDRICH) were added. The procedure was then continued as described above. For Endo H digestion, the beads were resuspended in 0.1 M sodium phosphate buffer (pH5.5) containing protease inhibitor as described above. The samples were divided into two and incubated for 15 minutes at room temperature with, or without 0.5 mU of Endo H (SIGMA).

### Flow Cytometri

Samples were analyzed using a FACS Calibur cytometer and FlowJo software (Tree Star Inc.)

### Immunofluorescense and Confocal Microscopy

Transfected M1 cells were grown to 50-70% confluence imaging dishes. Cells were then fixed with 3% paraformaldehyde, stained with indicated primary and secondary antibodies in 0.1% saponine and mounted onto object-glasses with Mowiol (Sigma-Aldrich, St Louis, Mo., USA). The microscope used was Olympus FV1000 confocal scanning laser upright microscope (BX61WI) with a PlanApo 60×/1.10 oil objective. Three channel PMT detector unit. Fluorochromes were exited with 488 nm Argon, 543 and 647 nm HeNe lasers. All image acquisition was done by sequential line scanning to eliminate bleed-through. Images were processed with ImageJ (NIH, USA).

### Results

### Biochemical Characterization of IiRRP/L17A

The Iiwt, sorting signal (QRD)L7I which aids in AP-1 and AP-2 binding was replaced by an (RRP) L7I motif by means of QuickChange PCR mutagenesis. This particular mutation was chosen based on previous work by Gupta et al. where it was found that RRP inserted into the Ii cytoplasmic tail bound AP-3 in vitro. Interaction with AP3 would ensure direct sorting to endolysosomal compartments. As a negative control of Ii trafficking, we employed the double leucine mutant IiL7A/L17A known to accumulate at the cell surface. The Ii constructs used in this study are illustrated in FIG. 1A.

In order to function as a chaperone for MHC II antigen presentation, Ii depends on two important features; the ability to trimerize[17] and to traffic to the endosomal pathway[18, 19]. The trimerization of IiRRP/L17A was analyzed by western blotting (WB). FIG. 1B shows that all constructs are able to form trimers to a similar extent yet, differences in monomers are observed.

Introducing the RRP amino acid sequence upstream of the first L718 sorting signal in the Ii tail (MDDRRPL7I (SEQ ID NO:39)) revealed a putative ER retention signal, namely the double arginine motif. As we observed a difference in the monomer levels (FIG. 1A) for the Ii proteins, we wanted to exclude the possibility that IiRRP/L17A was trapped in the ER. To this end, we performed an Endoglycosidase H (Endo H) treatment. If this Ii mutant traversed the Golgi Network prior to endosomal sorting, it would have acquired Endo H resistance due to addition of complex carbohydrate moieties on its two possible N-linked glycans in position 113 and 119. Indeed, three Ii fractions were detected for all samples (FIG. 1C) representing; the light/Endo H sensitive ER fraction, the premature Endo H resistant fraction with only one glycan and the fully mature double N-linked glycosylated mature fraction of Ii. Thus, all contructs gained Endo H resistance indicating that despite the presence of the RRP amino acid sequence, regular transport from ER through the Golgi was retained (FIG. 1 C).

Targeting of IiRRP/L17A to the late endosomes/lysosomes was also expected to reduce the half-life (t½) of this protein compared to the wt which traffics via the cell surface and in addition induces delayed endosomal maturation. In order to assess the t½ of the protein, we carried out a pulse-chase experiment.

Transfected cells were pulsed with with 35Met/35Cys containing media, and chased for various time points, followed by an immunoprecipitation of Ii. As seen in FIG. 1 D the T½ of Iiwt is approximately 3 hours, whereas IiRRP/L17A has a half-life closer to 1 hour, which suggests a faster transport to more proteolytic late endosomal compartments. As a control, IiL7A/L17A was found to accumulate within the four hours of chase due to inhibited internalization (Simonsen et al 1993). That the IiRRP/L17A is transported to late proteolytic compartment is corroborated by the data in FIG. 1 E showing that treatment of the cells with a Cathepsin S inhibitor and the broad protease inhibitor, Leupeptin. This mutant was no longer degraded, but accumulated, and to a higher degree than the wild type Ii and Ii L7A/L17A (FIG. 1E).

In further support for late endosomal localization of IiRRP/L17A, we show that when transfected cells were treated with Cathepsin S inhibitor and the broad protease inhibitor, Leupeptin, the mutant displayed the strongest protection from degradation (FIG. 1E).

We designed IiRRP/L17A to follow an AP3 sorting pathway. While AP1 and -2 are located at the TGN and plasma membrane, AP3 is involved in binding to and sorting of protein at TGN and early endosomes, such as LIMPII, bringing them to late endosomes/lysosomes, and is thus involved in endosomal maturation. We therefore performed RNAi mediated depletion of AP3 to further investigate the effect on the Ii protein levels. As many protolytic enzymes also traffic via an AP3 route to late endosomes/lysosomes one might expect that wild type invariant chain is effected. A mutant molecule which is also dependent on AP3 trafficking should then be more influenced. As shown in FIG. 1F, AP3 depletion resulted in accumulation of both Iiwt and IiRRP/L17A, however, IiRRP/L17A was protected against degradation to a higher extent than Iiwt, suggesting that the AP3 depletion interfered with trafficking of this Ii mutant. Thus, IiRRP/L17A trimerizes and exits ER similar to Iiwt, yet differs by being dependent on AP3 for trafficking to late endosomal compartments and having a shorter half-life.

### Subcellular Characterization of Ii RRP/L17A

The subcellular distribution of IiLRRP/L17A was further investigated by confocal imaging analysis. M1 cells were transfected with Ii wt and mutants as indicated (FIG. 2). The cells were incubated with the IgM anti Ii C-terminal antibody Bu43 for 30 min prior to staining with anti-Lamp and Pin-1, an Ii antibody which binds the cytoplasmic tail of Ii. The Ii wt, Ii L7A and the Ii L17A constructs were located at the plasma membrane and throughout the endosomal pathway supporting that one intact leucine signal is sufficient for Ii sorting (FIG. 2 A-C). However Ii L7A/L17A, was permanently located at the plasma membrane unable to internalize as the sorting signals were removed (FIG. 2 D). This was also expected and has previously been described for Ii constructs were the Ii N-terminal tail was fused to neuraminidase[15]. We still observed some cells with Bu43 positive endosomes most likely due to constitutive membrane internalization/recycling.

In addition, four putative AP-3 binding Ii molecules were made, and their cellular distribution examined. As for Iiwt; Ii L7A RAP, Ii L7A RRP and Ii RAP L17A all had internalized Bu43, indicating that they reached the cell surface (FIG. 2 E-G). They were also found in endosomes positive for Lamp-1, suggesting that after internalization these endosomes had matured into late endosomes/lysosomes. Ii L17A RAP had, however fewer and smaller ILEVs when compared to the Ii wt, indicating that the mutations had some effect on the cellular distribution (FIG. 2 G). IiL17A RRP turned out to be the most interesting mutant. Cells expressing this protein had highly reduced Bu43 labeling at the cell surface, but Ii cytoplasmic tails were found to co-localize with Lamp-1. Co-localization between Pin-1 and Lamp-1 devoid of Bu43 strongly indicated that a fraction of Ii L17A RRP is targeted directly to Lamp-1 positive compartments without ever reaching the cell surface (FIG. 7H).

### Cells Expressing Ii Carrying Tumor-Associated Epitopes Efficiently Load HLA-A2 and Specifically Activate CD8+ T Cells

We and others have recently shown that Iiwt can associate with MHC class I and mediate trafficking of MHC class I to the endosomal pathway[14] [12]. Basha and colleagues suggested that Ii might have an important role in cross presentation [12], whereas we showed that Ii in which CLIP was replaced by known CTL epitopes from the cancer targets MART-1 or CD20 efficiently activated antigen specific cytotoxic T cells (CTLs) when expressed in HLA-A2 positive cells. We therefore tested the ability of the IiL17A/RRP mutant to load HLA-A2 peptides when CLIP in this mutant was replaced by MART-1 (FIG. 3A). J76 cells stably expressing MART-1 specific TcR (DMF5) were incubated with HLA-A2 positive presenting cells expressing different Ii constructs. IL-2 secretion was used as a read-out for specific TcR stimulation. As a control, cells expressing HLA-A2 single-chain trimer (SCT) combined with MART-1 peptide or an irrelevant peptide (SCT-M1 and SCT-irr, respectively) were used. In this case the presenting cells are expressing a unique peptide-HLA-A2 molecule, which represents a saturated positive control. As shown, only the combination of HLA-A2 loaded with MART-1 peptide was able to stimulate DMF5. Interestingly, when MART-1 peptide was loaded with IiL17A/RRP, the intensity of the stimulation was almost equal to the saturating stimulation observed with SCT-M1. Similar results were observed with other TcRs (supp. Data: CD20 for the patent ONLY, but TGFbRII for the paper). In order to confirm that the signal observed was due to an increase peptide loading generated by the IiL17A/RRP, we stained the cells with the soluble form of DMF5. As shown in FIG. 3B, soluble DMF5 was able to stain cells expressing SCT-MART-1 (100%, MFI: 3.7×104 vs. 80 for the negative control). As reported previously (REF), soluble TcRs (sTcRs) like TcR are low affinity antigen-specific protein, therefore the detection of endogenous protein revealed difficult. However although the staining of Iiwt MART-1 expressing cells was low (2%, MFI: 105) cells expressing Ii L17A/RRP MART-1 were clearly detected (21%, MFI: 194), suggesting that the peptide loading was increased with this mutant. Similar results were obtained using CD20 sTcR (DATA ONLY FOR THE PATENT), where cells expressing IiL17A/RRP_CD20 (60%) were almost as efficiently recognized as cells expressing the SCT)-CD20 (94%) and to much higher extent than Iiwt-CD20. Together these data support the proposition that Ii RRP/L17A improves the loading of peptide placed in Ii-CLIP region.

### Discussion

We and others have recently shown that Iiwt can associate with MHC class I and mediate trafficking of MHC class I to the endosomal pathway [12]. Basha and colleagues suggested that Ii might have an important role in cross presentation [12] [14], whereas we showed that Ii in which CLIP was replaced by known CTL epitopes from the cancer targets MART-1 or CD20 efficiently activated antigen specific cytotoxic T cells (CTLs) when expressed in HLA-A2 positive cells. More importantly, we further found that activation of CTLs using this Ii-CLIP replaced strategy was independent of the transporters associated with antigen presentation (TAP) and the proteasome, facilitating novel vaccination strategies against cancer [14]. Here we took the strategy a step further by introducing specific point mutations within the cytoplasmic tail of Ii. Ii is known to depend on the adaptor proteins AP1 and AP2 for proper trafficking from Golgi, via the cell surface to the endosomal pathway [1-5]. By introducing AP3 binding motifs within the Ii tail of a TfR-Ii fusion construct, Bakke and colleagues has previously shown evidence for direct sorting to late endosomal/lysosomal structures in cells [15]. Here we show that full length Ii harbouring residues favouring AP3 binding, is also re-routed to late endosomes from the Golgi, gain shorter T½ and colocalise with lysosomal markers to a higher extent than Iiwt.

Interestingly, neither MHC I or MHC II is directly dependent on AP3 for proper trafficking and antigen presentation. It was reported that the kinetics of Ii transport and degradation is unaffected by the lack of AP3 [27]. In addition, phagosomal maturation in DCs has been found to proceed normally in AP3 deficient mice [28]. In this study, we find that AP3 depletion leads to an accumulation of both Iiwt and IiRRP/L17A, however the effect on the AP3 mutant is significantly higher. This indicates that the accumulation of Ii proteins in AP3 depleted cells in this study is mostly due to impaired trafficking.

We further confirm previous results showing that Iiwt trafficking through the endosomal pathway is delayed. After 1 hour incubation with the Ii specific antibody M-B741, we find the antibody mainly in early Rab5 positive compartments, whereas little is found in Rab7 positive compartments

### Ap3 Mutants:

AP3 binds both leucine ([DE]xxxL[LI]) and tyrosine (YxxØ) based sorting motifs through their β- and μ-chain respectively (Craig H M Virology 2000), Dell'Angelica EC EMBO J 1997). The residues N terminal to the two (iso)leucines of the sorting signal determine adaptor binding (Rodionov 2002) and certain residues favor AP3 binding. LIMPII is a well known AP3 binder harboring a RAP motif in front of the LI signal. AP3 is required for mouse CD1d (RRRSAYQDIR (SEQ ID NO:29)) mediated antigen presentation of glycosphingolipids to NKT cells (Elewaut D 2003, JEM and Lawton A P, JI 2005). Human CD1b (RRRSYQNIP (SEQ ID NO:30)) is the only human CD molecule being dependent on AP3 for proper trafficking and antigen presentation (Sugita M, Immunity 2002). These molecules harbor tyrosin based sorting motifs, however the requirement for positively charged Arginine residues seems to be similar to both leucine—and tyrosine based sorting signals. Whether upstream residues are important for AP3 binding to tyrosin signals is not investigated, yet not unlikely.

Whether Lysines can replace Arginines are currently unknown.

Surface plasmon resonance experiments using a GST-tagged cytoplasmic tail of Ii has shown that AP3 motifs in front of both L7 and L17 mediate binding to AP3 in vitro (Rodionov 2002). AP3 binding motifs in an Ii-TfR chimera have only been investigated in front of the L7 signal, and here RRR/L17A were found to give the most efficient subcellular redistribution (Gupta et al 2006). However, several mutants could be tested in the antigen presentation assay in order to create different targeting efficiencies.

Below is a table containing all the mutants tested by SRP or in MDCK from Bakke and co-workers:

| | AP3 | | |
|---|---|---|---|
| | binding | | |
| Mutant | (SPR) | Functional assay | ref |
| L17A | 0% | GST/liTfR chimera | Gupta SN 2006, |
| | | | Rodionov D 2002 |
| L17A, QRD→RAP* | 25% | GST/liTfR chimera | Gupta SN 2006, |
| | | | Rodionov D 2002 |
| L17A, QRD→RRP* | 55% | GST/liTfR chimera | Gupta SN 2006, |
| | | | Rodionov D 2002 |
| L17A, QRD→QAP | 13% | GST/liTfR chimera | Gupta SN 2006, |
| | | | Rodionov D 2002 |
| L17A, QRD→RAD | 25% | GST/liTfR chimera | Gupta SN 2006, |
| | | | Rodionov D 2002 |
| L17A, QRD→QRP | 14% | GST/liTfR chimera | Gupta SN 2006, |
| | | | Rodionov D 2002 |
| L17A, QRD→QAD | 4% | GST/liTfR chimera | Gupta SN 2006, |
| | | | Rodionov D 2002 |
| L17A, QRD→RRD | 0% | GST/liTfR chimera | Gupta SN 2006, |
| (D6R) | | | Rodionov D 2002 |
| L7A | | GST | Rodionov D 2002 |
| L7A, QLP→RAP* | ~10% | GST | Rodionov D 2002 |
| L7A, QLP→RLP | ~22% | GST | Rodionov D 2002 |
| L7A, QLP→QAP | ~20% | GST | Rodionov D 2002 |
| L7A, QLP→RRP* | ~60% | GST | Rodionov D 2002 |
| L7A, | ~80% | GST | Rodionov D 2002 |
| NEQLP→DERAP | | | |

Gupta and Rodinov made GST tagged Ii which was tested for AP3 binding by Surface Plasmon Resonance. Mostly, they used the Ii cytoplasmic tail, therefore, it was in our interest to test this out for the full length protein.

### Example 2—TGFbRII Frameshift Peptide

This example is a combination the invariant chain technology with the TGFbRII frameshift peptide, and analysis of the immunogenicity of the construct when presented to specific TCR expressing Tcells.

Two anti-TGFbRII peptide specific TCRs were used (Radium Cys and Radium wt). The activity was measured by CD107 detection on Tcells which is a marker for cytotoxic vesicles. Cells expressing li-TGFbRII or li-wt (control) in combination with HLA-A2 were used as APC. The inventors used single-chain trimmers (SCT), which mimics a full peptide loaded cell expressing TGFbRII peptide (SCT-TGFbRII) as a positive control and MART-1 peptide (SCT-M1) as a negative control.

Finally, the same samples were also tested with Tcells expressing MART-1 specific TCR (medium grey) as a specificity control. The results can be seen in FIG. 7.

This experiment shows that the TGFbRII frameshift peptide can be presented by HLA-A2 and recognized by specific Tcells.

The constructs used:

(SEQ ID NO: 3)

MDDQRDLISNNEQLPMLGRRPGAPESKCSRGALYTGFSILVTLLLAGQA
TTAYFLYQQQGRLDKLTVTSQNLQLENLRMKLPKPPKPVSKRLSSCVPV
AQALPMGALPQGPMQNATKYGNMTEDHVMHLLQNADPLKVYPPLKGS
FPENLRHLKNTMETIDWKVFESWMHHWLLFEMSRH
SLEQKPTDAPPKESLELEDPSSGLGVTKQDLGPVPM

TGFbRIIp is underlined in the above sequence

(SEQ ID NO: 4):
RLSSCVPVA
(SEQ ID NO: 6)
MDDQRDLISNNEQLPMLGRRPGAPESKCSRGALYTGFSILVTLLLAGQ
ATTAYFLYQQQGRLDKLTVTSQNLQLENLRMKLPKPPKPVSKKSLVRL
SSCVPVALMSAMTQALPMGALPQGPMQNATKYGN
MTEDHVMHLLQNADPLKVYPPLKGSFPENLRHLKNTMETIDWKVFESW
MHHWLLFEMSRHSLEQKPTDAPPKESLELEDPSSGLGVTKQDLGPVPM

TGFbRIIp4-1 is underlined in the above sequence

(SEQ ID NO: 7):
KSLVRLSSCVPVALMSAMT
(SEQ ID NO: 8)
MDDQRDLISNNEQLPMLGRRPGAPESKCSRGALYTGFSILVTLLLAGQ
ATTAYFLYQQQGRLDKLTVTSQNLQLENLRMKLPKPPKPVSKSLVRLS
SCVPVALMSAMTTSSSQQALPMGALPQGPMQNATKYGN
MTEDHVMHLLQNADPLKVYPPLKGSFPENLRHLKNTMETIDWKVFES
WMHHWLLFEMSRHSLEQKPTDAPPKESLELEDPSSGLGVTKQDLGPVPM

TGFbRIIp4-2 is underlined in the above sequence

(SEQ ID NO: 9):
SLVRLSSCVPVALMSAMTTSSQ

The corresponding DNA sequences were cloned and tested with the TGFbRII TCR in a similar assay as shown above: The Radium TCR expressing cells were incubated with APCs expressing SCT-TGFbRIIp as control or the 3 sequences given above. CD107 expression was used as readout. These results can be seen in FIG. 8.

This experiment shows that the long peptides p4-1 and p4-2 are processed and presented by HLA-A2.

### Example 3—Wt Invariant Chain with Frameshift TGFβRII

The experiments of example 2 were used as basis for a clinical tests.

### Material and Methods

### Patients

Thirteen subjects with surgically treated colorectal cancer Dukes's C (after cytotoxic treatment) or Duke's D were enrolled between October 2000 and May 2002.

Inclusion criteria: surgically treated colorectal cancer Dukes's C (after cytotoxic treatment) or Duke's D. MIN phenotype verified by standard test for microsatellite instability (BAT-26) age ≥18 and ≤75 years, The clinical trial was approved by the Norwegian Medicines Agency, the Committee for Medical Research Ethics, Region South and the Hospital Review Board and performed in compliance with the World Medical Association Declaration of Helsinki. Written informed consent was obtained from the patients.

### Vaccine

The peptide vaccine consisted of equimolar concentrations of three different freeze-dried frameshift peptides. One of the peptides, (P01-2602) KSLVRLSSCVPVALMSAMT (SEQ ID NO: 7), reflects an amino acid sequence expressed by a frameshift mutated TGFβRII gene.

### Generation of T-Cell Lines and Clones Specific for TGFβRII Frameshift Peptides

PBMCs were obtained pre-vaccination and post-vaccination (week 6).

Pre- and post-vaccination samples were analyzed in parallel for proliferative response to peptide vaccine stimulation (3H-Thymidine incorporation assays). The PBMCs had been isolated and frozen. In brief, 50 ml of Acid Citrate Dextrose-blood was collected and PBMCs were isolated 0.5-3 hours later by centrifugation over Lymphoprep (Axis-Shield).

The PBMCs were frozen in RPMI-1640 (PAA Laboratories) with 35% human serum (HS, PAA Laboratories) and 10% dimethyl sulfoxide (DMSO, (Sigma-Aldrich)) for storage in liquid nitrogen. Viability of cells upon thawing was found to be between 94-97% as assessed by the trypan blue exclusion test. Thawed PBMCs were stimulated once in vitro with peptide at 2×106 cells/ml in RPMI-1640 medium containing 10% human serum (HS) (Blood bank, Haukeland Hospital Bergen, Norway). 10 U/ml IL-2 (Chiron) was added on day 2 of culture. For proliferation assays, T cells were harvested on day 10-12 and seeded in triplicates in 96-well U-bottomed microtiter plates, generally at 5 104 cells per well (c/w). The same number of irradiated (30 Gy), autologous PBMCs was added for use as APCs. TGFβRII frameshift peptides were added at 25. This included peptide 573 (p573), RLSSCVPVA (amino acid sequence 131-139), peptide 621 (p621), KSLVRLSSCVPVALMSAMT (SEQ ID NO: 7), and peptide 538 (p538), SLVRLSSCVPVALMSAMTTSSSQ (SEQ ID NO: 9), from a TGFβRII frameshift protein resulting from a 1 bp-deletion (−1A) in an adenosine stretch (A10) from base number 709-718 of TGFBRII (The GenBank sequence for wild type human TGFBRII: NM 003242). All peptides were provided by Norsk Hydro, ASA, Porsgrunn, Norway. Proliferation was measured on day 3 after overnight labelling with 3.7 104 Bq 3H-Thymidine (Laborel, Oslo, Norway) before harvesting. All peptides were manufactured by Norsk Hydro, Norway. The stimulation Index (SI) was defined as proliferation (counts per minute, cpm) with peptide divided by proliferation without peptide and an SI≥2 was considered a positive response. T-cell clones from responding T-cell lines were generated as previously described (Sæterdal et al, 2001).

### TcR Cloning

Frameshift-specific T-cell clones were grown and total RNA was prepared. The cloning was performed using a modified 5′-RACE method. Briefly, cDNA was synthesized using an oligo-dT primer and was tailed at the 5′-end with a stretch of cytosines. A polyguanosine primer together with a constant domain-specific primer was used to amplify TCR chains. The amplicon was cloned and sequenced. The expression construct was prepared by amplifying TCR-α and -β chains separately with specific primers and a second PCR was performed to fuse the TCR chains as a TCR-2A construct. The TCR-2A reading frame was cloned into pENTR (Invitrogen) and subsequently recombined into other expression vectors. For RNA synthesis we subcloned the insert into a Gateway modified version of pCIpA102 (Sæbøe-Larssen et al, 2002). A detailed method as well as the primers sequences can be found in Walchli et al. 2011.

### Ii Chain Constructs

Ii constructs were made by site direct mutagenesis of the original vector (pENTR) using primers as described in Walchli et al. 2014. For long peptide insertion, the CLIP sequence of the Ii WT was replaced by a linker containing 2 restriction sites (KpnI and Xhol) by site directed mutagenesis using these primers:

pWS-CLIPRS

(SEQ ID NO: 14)

GCTCGAGTGCTGCGGTACCCTTGCTCACAGGCTTGGG
and
pSW-CLIPRS

(SEQ ID NO: 19)

GGTACCGCAGCACTCGAGCAGAATGCCACCAAGTATGGC.

This construct was then used as starting material for subcloning of long peptide coding sequences that were ordered as long phosphorylated primers.

The two following sequences were ordered

TGFp4-1 (KSLVRLSSCVPVALMSAMT, SEQ ID NO:7) and
TGFp4-2 (SLVRLSSCVPVALMSAMTTSSSQ, SEQ ID NO: 9):
TGFp4-1uCaagagcctggtgagactgagcagctgcgtgcccgtggccc
tgatgagcgccatgaccC (SEQ ID NO: 15), TGFp4-11 TCGA
GGGTCATGGCGCTCATCAGGGCCACGGGCACGCAGCTGCTC
AGTCTCACCAGGCTCTTGGTAC (SEQ ID NO: 16), TGFp4-2u
Cagcctggtgagactgagcagctgcgtgcccgtggccctgatgagcgcca
tgaccaccagcagcagccagC (SEQ ID NO: 17) and TGFp4-21
TCGAGCTGGCTGCTGCTGGTGGTCATGGCGCTCATCAGGGCCA
CGGGCACGCAGCTGCTCAGTCTCACCAGGCTGGTAC
(SEQ ID NO: 18).

In brief, primer pairs were annealed and subcloned by T4 ligase into the pre-open and dephosphorylated CLIP-replaced construct. After sequence verification, the constructs were recombined into a Gateway modified version of pCIpA102 (Sæbøe-Larssen et al, 2002).

In Vitro mRNA Transcription of TCR and Ii Chain Constructs

In vitro mRNA synthesis was performed essentially as previously described (Almåsbak et al, 2011). Linearized DNA was purified with a Wizard® SV gel and polymerase chain reaction (PCR) clean-up system (Promega, Madison, Wis., USA) according to the manufacturer's instructions. The in vitro transcription (IVT) of the DNA template was performed using a Ribomax large-scale RNA production system-T7 (Promega) with a modified reaction mix containing increased concentrations of rGTP and cap analoges [3 mM rGTP, 9 mM m7G(5′)ppp(5′)G]. Following transcription, mRNA was extracted with Trizol/Cloroform (Invitrogen) according to the manufacturer's instructions, precipitated with isopropanol, washed with ethanol and dissolved in RNase-free water. The mRNA quality was checked by agarose gel electrophoresis and RNA concentration and purity were assessed by Nanodrop (Thermo Fisher Scientific, Waltham, Mass., USA).

### In Vitro Expansion of Human T Cells

T cells from healthy donors were expanded with using a protocol adapted for GMP production of T cells employing CD3:CD28 DYNABEADS™ reagent, essentially as previously described (Rasmussen et al, 2010). In brief, PBMCs were isolated from buffy coats by density gradient centrifugation, depleted of monocytes and cultured with CD3:CD28 DYNABEADS™ reagent (DYNABEADS™ CLINEXVIVO™ CD3/CD28 reagent, kindly provided by Dynal Invitrogen, Oslo, Norway) at a 1:3 ratio in complete CellGro DC Medium with 100 U/mL recombinant human interleukin-2 (IL-2) (Proleukin, Novartis Vaccines & Diagnostics Inc., Emeryville, Calif., USA) for 10 days. The T cells were used fresh or frozen and aliquots thawed and rested in complete medium before mRNA transfection.

### Electroporation of T Cells and EBV-LCL

Expanded T cells and Epstein-Barr virus-transformed lymphoblastoid cell lines (EBV-LCL) were washed twice and resuspended in CellGro DC medium (CellGenix GmbH) at 10-70×106 cells/mL. The mRNA was mixed with the cell suspension at 100 μg/mL, and electroporated in a 4-mm gap cuvette at 500 V and 2 ms using a BTX 830 Square Wave Electroporator (BTX Technologies Inc., Hawthorne, N.Y., USA). Immediately after transfection, T cells and EBV-LCL were transferred to complete culture medium at 37° C. in 5% CO2 overnight to allow TCR or Ii chain-TGFβRII peptide expression.

Functional Assays and CD4+ and CD8+-Cell Stimulation by Long TGFβRII Frameshift Peptide

For analysis of TCR expression, electroporated T cells were washed in staining buffer consisting of phosphate buffered saline (PBS) containing 0.1% human serum albumin (HSA) and 0.1% sodium azide before staining with anti-Vβ3-Fluorescein isothiocyanate (FITC) (Beckman Coulter-Immunotech SAS, Marseille, France), anti-CD4-Brilliant Violet (BV) 421 (BioLegend), CD4-Phycoerythrein (PE)-Cy7 (eBioscience, San Diego, USA), anti-CD8-BV 421 (BioLegend), CD8-PerCP-Cy5.5 (eBioscience) or CD8-PE-Cy7 (eBioscience) for 20 min at RT. The cells were then washed in staining buffer and fixed with 1% paraformaldehyde.

For intracellular staining, electroporated T cells or T cell clones were stimulated for 5 hours or overnight in the presence of BD GolgiPlug and BD Golgistop at a 1/1000 dilution with EBV-LCL lines as target cells electroporated or not with various Ii chain mRNA at a T-cell to target ratio of 1:2. Antigen presenting cells (EBV-LCLs HLA-A2+ and HLA-DR7+) electroporated with Ii-construct mRNA and then incubated with a CD4+ T cell clone previously isolated from a vaccinated HLA-DR7+ patient (the vaccination peptide was p621, KSLVRLSSCVPVALMSAMT (SEQ ID NO:40), corresponding to 4-1). HLA-A2+ EBV-LCL were used to stimulate T cells expressing TGFβRII frameshift mutation-specific TcR (Radiuml, which recognizes peptide RLSSCVPVA (SEQ ID NO:4) and is HLA-A*0201 restricted). As a control the presenting EBV-LCLs were either loaded with 5 μM of the vaccination peptide (long peptide, p621) or the short peptide (RLSSCVPVA (SEQ ID NO:4)). As a control for mRNA synthesis and electroporation, we also used a single-chain trimer construct expressing the short peptide (SCT-TGFbsp). Tc incubated with untreated EBV-LCLs were used as negative controls.

CD107a-PE-Cy5 was added for the last hour of the incubation. Cells were stained extracellularly using above-mentioned antibodies and intracellularly with IFN-γ-FITC (eBioscience), IL-2-APC (eBioscience) and TNF-α-PE using the PerFix kit (Beckman Coulter) according to the manufacturer's instructions. All antibodies and reagents for intracellular cytokine staining were purchased from BD Bioscience, New Jersey, USA except where noted. Cells were acquired on a BD LSR II flow cytometer and the data were analysed using FlowJo software (Treestar Inc., Ashland, Oreg., USA).

Gating strategy: In this example, gates were set on CD4+ Tc and the presence of IFN-γ or TNF-α was monitored. The percentage of positive cells is reported on the graph shown in results section.

### Results

As shown the CD4+ Tc responded to both Ii chain constructs with long peptides (Ii TGFp4-1 and Ii TGFp4-2), in addition to the synthetic one (p621). As expected, the SCT with the short peptide (SCT-TGFbsp) did not stimulate the CD4+ T cells. As previously shown, CD8+Tc also recognized the long constructs in addition to the SCT. The results can be seen in FIGS. 9-10.

### CONCLUSION

the use of long peptide in combination to Ii is able to evoke both CD4+ and CD8+ T cell responses and can thus induce a broader immune response required for T-cell memory responses and thus expected to be more clinically beneficial. Moreover, the long peptides have been shown to induce immune responses in patients with a wide range of HLA genotypes and can therefore be used in a much larger patient population than the short, HLA-A*0201 restricted peptide.

1. A nucleic acid molecule encoding a type II transmembrane invariant chain (Ii) which is modified by exchanging the class II-associated Ii peptide (CLIP) with an antigen that activates both CD4+ and CD8+ T cells.

2. The nucleic acid molecule according to claim 1, in which the Ii is wildtype Ii.

3. The nucleic acid molecule according to claim 1, in which the antigen is selected from the group consisting of TGFbRIIp, TGFbRIIp4-1, TGFbRIIp4-2, TGFp4-1, and TGFp4-2.

4. The nucleic acid molecule according to claim 1, wherein the nucleic acid molecule is selected from the group consisting of mRNA and DNA.

5. The nucleic acid molecule according to claim 4, wherein the nucleic acid molecule is mRNA.

6. A polypeptide comprising the type II transmembrane invariant chain (Ii) described in claim 1.

7. A method of presenting a CD8+ and/or CD4+ T-cell activating antigen on a cell, comprising:
a) modifying the type II transmembrane invariant chain (Ii) by exchanging the class II-associated Ii peptide (CLIP) with an antigen,
b) introducing the Ii to a cell.

8. The method according to claim 7, wherein the antigen is a tumor antigen.

9. The method according to claim 7, wherein the antigen is selected from the group consisting of TGFbRIIp, TGFbRIIp4-1, TGFbRIIp4-2, TGFp4-1, and TGFp4-2.

10. A pharmaceutical composition comprising the nucleic acid molecule according to claim 1 and a pharmaceutically acceptable carrier, excipient and/or diluent.

11. The pharmaceutical composition according to claim 10, which is a vaccine.

12. A method for inducing a CD8+ and/or a CD4+ response, comprising administering the nucleic acid molecule according to claim 1 to an individual.

13. The method according to claim 12, in which the nucleic acid molecule is administered using electroporation.

14. A pharmaceutical composition comprising the polypeptide according to claim 13 and a pharmaceutically acceptable carrier, excipient and/or diluent.

15. The pharmaceutical composition according to claim 14, which is a vaccine.

16. A method for inducing a CD8+ and/or a CD4+ response, comprising administering the polypeptide according to claim 6 to an individual.

17. The method according to claim 16, in which the polypeptide is administered using electroporation.


SEQUENCE LISTING (40 sequences)

SEQ ID NO: 1 - protein, 216 aa, Homo sapiens
MDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 2 - protein, 9 aa, Artificial Sequence
  synthetic
MRMATPLLM

SEQ ID NO: 3 - protein, 216 aa, Artificial Sequence
  synthetic
MDDQRDLISNNEQLPMLGRRPGAPESKCSRGALYTGFSILVTLLLAGQATTAYFLYQQQGRLDKLTVTSQNLQLENLRMKLPKPPKPVSKRLSSCVPVAQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 4 - protein, 9 aa, Artificial Sequence
  synthetic
RLSSCVPVA

SEQ ID NO: 5 - protein, 216 aa, Artificial Sequence
  synthetic
MDDRRPLISNNEQLPMAGRRPGAPESKCSRGALYTGFSILVTLLLAGQATTAYFLYQQQGRLDKLTVTSQNLQLENLRMKLPKPPKPVSKRLSSCVPVAQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 6 - protein, 226 aa, Artificial Sequence
  synthetic
MDDQRDLISNNEQLPMLGRRPGAPESKCSRGALYTGFSILVTLLLAGQATTAYFLYQQQGRLDKLTVTSQNLQLENLRMKLPKPPKPVSKKSLVRLSSCVPVALMSAMTQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 7 - protein, 19 aa, Artificial Sequence
  synthetic
KSLVRLSSCVPVALMSAMT

SEQ ID NO: 8 - protein, 230 aa, Artificial Sequence
  synthetic
MDDQRDLISNNEQLPMLGRRPGAPESKCSRGALYTGFSILVTLLLAGQATTAYFLYQQQGRLDKLTVTSQNLQLENLRMKLPKPPKPVSKSLVRLSSCVPVALMSAMTTSSSQQALPMGALPQGPMQNATKYGNMTEDHVMHLLQNADPLKVYPPLKGSFPENLRHLKNTMETIDWKVFESWMHHWLLFEMSRHSLEQKPTDAPPKESLELEDPSSGLGVTKQDLGPVPM

SEQ ID NO: 9 - protein, 22 aa, Artificial Sequence
  synthetic
SLVRLSSCVPVALMSAMTTSSQ

SEQ ID NO: 10 - DNA, 33 bp, Artificial Sequence
  synthetic
agagagggtaccgtcatggatgaccagcgcgac

SEQ ID NO: 11 - DNA, 33 bp, Artificial Sequence
  synthetic
agagagggatcctcacatggggactgggcccag

SEQ ID NO: 12 - DNA, 39 bp, Artificial Sequence
  synthetic
ccgtcatggatgaccgtcgtccccttatctccaacaatg

SEQ ID NO: 13 - DNA, 39 bp, Artificial Sequence
  synthetic
cattgttggagataaggggacgacggtcatccatgacgg

SEQ ID NO: 14 - DNA, 37 bp, Artificial Sequence
  synthetic
gctcgagtgctgcggtacccttgctcacaggcttggg

SEQ ID NO: 15 - DNA, 59 bp, Artificial Sequence
  synthetic
caagagcctggtgagactgagcagctgcgtgcccgtggccctgatgagcgccatgaccc

SEQ ID NO: 16 - DNA, 67 bp, Artificial Sequence
  synthetic
tcgagggtcatggcgctcatcagggccacgggcacgcagctgctcagtctcaccaggctcttggtac

SEQ ID NO: 17 - DNA, 71 bp, Artificial Sequence
  synthetic
cagcctggtgagactgagcagctgcgtgcccgtggccctgatgagcgccatgaccaccagcagcagccagc

SEQ ID NO: 18 - DNA, 79 bp, Artificial Sequence
  synthetic
tcgagctggctgctgctggtggtcatggcgctcatcagggccacgggcacgcagctgctcagtctcaccaggctggtac

SEQ ID NO: 19 - DNA, 39 bp, Artificial Sequence
  synthetic
ggtaccgcagcactcgagcagaatgccaccaagtatggc

SEQ ID NO: 20 - protein, 30 aa, Artificial Sequence
  synthetic
MDDQRDAISNNEQLPMLGRRPGAPESKCSR

SEQ ID NO: 21 - protein, 30 aa, Artificial Sequence
  synthetic
MDDQRDLISNNEQLPMAGRRPGAPESKCSR

SEQ ID NO: 22 - protein, 30 aa, Artificial Sequence
  Synthetic
MDDQRDAISNNEQLPMAGRRPGAPESKCSR

SEQ ID NO: 23 - protein, 30 aa, Artificial Sequence
  synthetic
MDDQRDAISNNERAPMLGRRPGAPESKCSR

SEQ ID NO: 24 - protein, 30 aa, Artificial Sequence
  synthetic
MDDQRDAISNNERRPMLGRRPGAPESKCSR

SEQ ID NO: 25 - protein, 30 aa, Artificial Sequence
  synthetic
MDDRAPLISNNEQLPMLGRRPGAPESKCSR

SEQ ID NO: 26 - protein, 30 aa, Artificial Sequence
  Synthetic
MDDQRDLISNNEQLPMLGRRPGAPESKCSR

SEQ ID NO: 27 - protein, 30 aa, Artificial Sequence; identical to SEQ ID NO: 22
  Synthetic

SEQ ID NO: 28 - protein, 30 aa, Artificial Sequence
  Synthetic
MDDRRPLISNNEQLPMAGRRPGAPESKCSR

SEQ ID NO: 29 - protein, 10 aa, Artificial Sequence
  Synthetic
RRRSAYQDIR

SEQ ID NO: 30 - protein, 9 aa, Artificial Sequence
  Synthetic
RRRSYQNIP

SEQ ID NO: 31 - protein, 5 aa, Artificial Sequence
  Synthetic
DERAP

SEQ ID NO: 32 - protein, 5 aa, Artificial Sequence
  Synthetic
QLPLI

SEQ ID NO: 33 - protein, 5 aa, Artificial Sequence
  Synthetic
RRPLI

SEQ ID NO: 34 - protein, 5 aa, Artificial Sequence
  Synthetic
QRDLA

SEQ ID NO: 35 - protein, 9 aa, Artificial Sequence
  Synthetic
MRMATPLLM

SEQ ID NO: 36 - protein, 9 aa, Artificial Sequence
  Synthetic
SLFLGILSV

SEQ ID NO: 37 - protein, 10 aa, Artificial Sequence
  Synthetic
ELAGIGILTV

SEQ ID NO: 38 - protein, 8 aa, Artificial Sequence
  Synthetic
MDDRRPLI

SEQ ID NO: 39 - protein, 19 aa, Artificial Sequence
  Synthetic
KSLVRLSSCVPVALMSAMT

SEQ ID NO: 40 - protein, 5 aa, Artificial Sequence
  Synthetic
RRPLA